(19) United States
(12) Plant Patent Application Publication
(10) Pub. No.: US 2003/0033653 P1
(43) Pub. Date: Feb. 13, 2003

(54) PETUNIA PLANT NAMED 'KEIYEUL'

(76) Inventors: Hiroshi Hirabayashi, Yachiyo-shi (JP); Eriko Hirabayashi, Yachiyo-shi (JP); Minako Hirabayashi, Yachiyo-shi (JP); Kanako Hirabayashi, Yachiyo-shi (JP)

Correspondence Address: KLARQUIST SPARKMAN, LLP, 121 SW SALMON STREET, SUITE 1600, PORTLAND, OR, 97204, US

(21) Appl. No.: 09/929,136

(22) Filed: Aug. 13, 2001

(51) Int. Cl. A01H 5/00

(52) U.S. Cl. PLT/356

(57) ABSTRACT

Petunia plant having a vigorous habit and profuse flowering of very light yellow colored flowers.

The present invention relates to a new and distinct variety of petunia which was originated by me by crossing an unnamed seedling (unpatented) with an unnamed seedling (unpatented), both of which were progeny of unpatented, unnamed seedlings.

### BACKGROUND OF THE INVENTION

The primary objective of this breeding was to produce a new petunia variety having large, single, yellowish color flowers; a free flowering, pendant habit; and free branching. The objective was substantially achieved, along with other desirable improvements, as evidenced by the following unique combination of characteristics that are outstanding in the new variety and that distinguish it from its parents, as well as from all other varieties:

1. Vigorous habit; and

2. Profuse flowering.

### DETAILED DESCRIPTION OF THE INVENTION

Asexual reproduction of this new variety by cuttings, as performed at Yachigo Farm Keisei Rose Nurseries, Inc., 755 Owadashinden, Yachiyo-shi, Chiba-Ken, Japan and at Somis, California, shows that the foregoing and all other characteristics and distinctions come true to form and are established and transmitted through succeeding propagations.

The accompanying illustrations show a typical specimen of this new variety, depicted in color as nearly true as it is reasonably possible to make the same in a color illustration of this character:

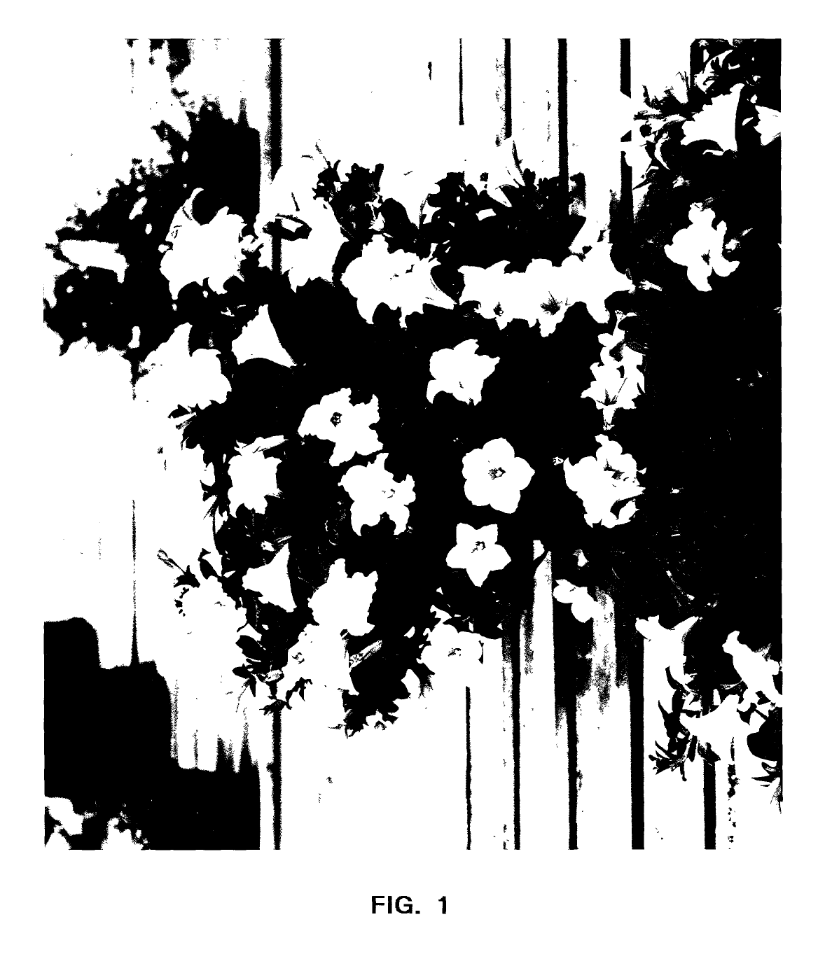

FIG. 1 is a photograph of a mature plant; and

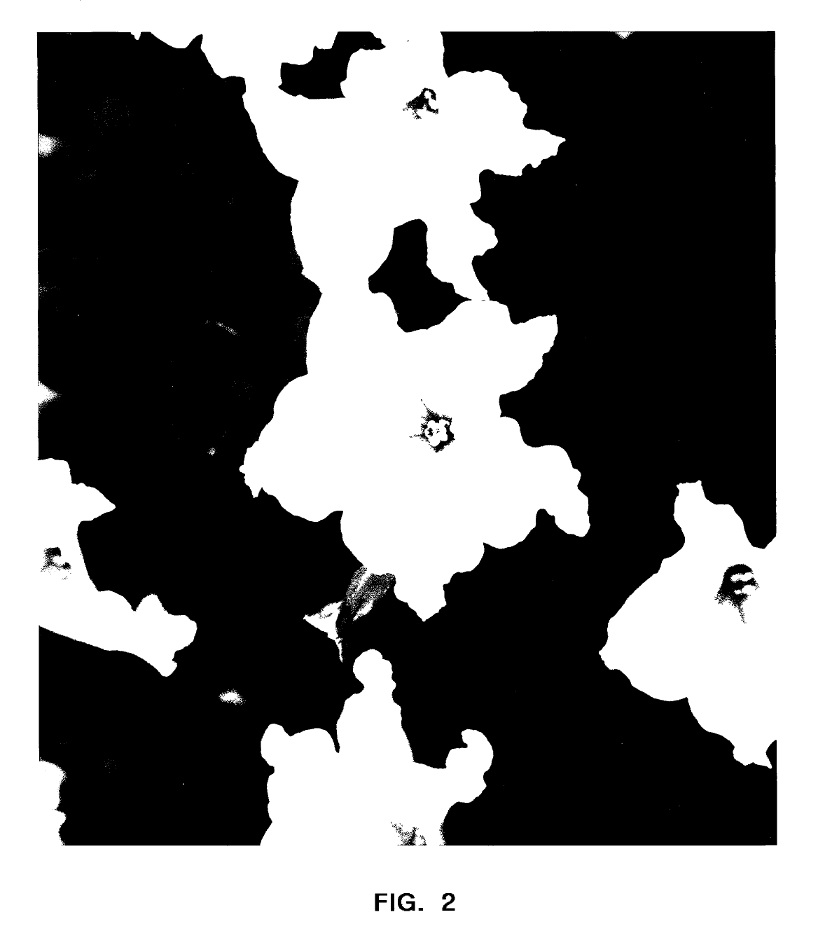

FIG. 2 is an enlarged view of a single flower and portions of adjacent flowers.

The following is a detailed description of my new petunia cultivar with color descriptions using terminology in accordance with The Royal Horticultural Society (London) colour chart, except where ordinary dictionary significance of color is indicated.

Parentage:

Seed Parent.—Unnamed seedling.

Pollen Parent.—Unnamed seedling.

Classification:

Botanical.—Petunia hybrida.

Commercial.—Petunia.

### FLOWER

Observations made from specimens grown in a garden environment at Somis, Calif. in July, 2000 to December, 2000.

Growth.—Spreading.

Growth habit.—Vigorous, spreading.

Mature Plant.—Height: 11-15 cm.

Spreading area.—50-75 cm in diameter.

Blooming Period.—From time day length exceeds twelve hours to first frost.

Stem.—Overall Length: 11-20 cm. Main Stem: 20 cm Laterals: 11-15 cm Thickness: 2-3 mm Color: Yellow Group 9D Pubescence: Medium Mode of Branching: Abundant Length of Internode: 2.20-2.50 cm (short)

Leaf.—Arrangement on stem: Slightly upward Shape: Elliptic. Size: 3.5-5.0 cm Thickness: About 1 mm. Color: Upper Surface: Yellow-Green Group 144A Lower Surface: Yellow-Green Group 144A Phyllotaxis: Perpendicular Pubescence: Medium.

Flower:

Orientation at Opening.—Slantly upward.

Size.—2-2 ⅜inches.

Type.—Single.

Shape.—Corolla is thin.

Color.—The upper sides of the petals are White Group 115 with five prominent veins of Yellow-Green Group 145B. The base of the throat of a flower is Yellow-Green Group 145A. The reverse sides of the petals are White Group 145A with five prominent veins of Yellow-Green Group 145C. The base of the throat is Yellow Green Group 145C.

Peduncle.—Length: 1.3-1.8 cm. Color: Yellow-Green Group 146C.

Calyx.—Number of sepals: 5. Length: 15-17 cm. Color: Yellow-Green Group 144A.

Reproductive parts:

Stamens.—Number: Five

Anthers.—Color: Yellow-White Group 158B.

Filaments.—Color: White Group 155A.

Pistil.—Quantity: One.

Stigma.—Color: Green Group 143A.

Style.—Color: Yellow-Green Group 149C.

Disease resistance: Resistant to mildew under normal growing conditions.

Response to climatic conditions: Hardy to Zone 10.

I claim:

1. A new and distinct variety of petunia, substantially as herein shown and described, characterized particularly by its vigorous habit and profuse very light yellow colored flowers.
